(19) United States
(12) Patent Application Publication
(10) Pub. No.: US 2020/0281567 A1
(43) Pub. Date: Sep. 10, 2020

(54) Method for Motion Correction of 3D Contrast Enhanced Ultrasound Without the Availability of Bmode Data

(71) Applicant: The Board of Trustees of the Leland Stanford Junior University, Stanford, CA (US)

(72) Inventors: Dimitre H. Hristov, Stanford, CA (US); Ahmed El Kaffas, Palo Alto, CA (US)

(21) Appl. No.: 16/809,889

(22) Filed: Mar. 5, 2020

Related U.S. Application Data
(60) Provisional application No. 62/814,054, filed on Mar. 5, 2019.

(51) Int. Cl. A61B 8/08

(52) U.S. Cl. CPC A61B 8/483; A61B 8/5276; A61B 8/481

(57) ABSTRACT

The present invention provides a validated method for motion correction with great potential for mitigating motion artifacts in 3D DCE-US. The method is described as a method for motion correction of three-dimensional contrast enhanced ultrasound without the availability of Bmode data. Four-dimensional cine data including three-dimensional contrast enhanced ultrasound image frames are acquired. The acquired three-dimensional contrast enhanced ultrasound image frames are subdivided into groups of similar images referred to as windows. A first pass registration is performed for each of the images in the window to a window representative image. A second pass is performed for each of the registered images from the first pass to a master reference image.

### CROSS-REFERENCE TO RELATED APPLICATIONS

This application claims priority from U.S. Provisional Patent Application 62/814,054 filed Mar. 5, 2019, which is incorporated herein by reference.

### FIELD OF THE INVENTION

This invention relates to methods, devices and systems for motion correction due to contrast fluid in medical imaging.

### BACKGROUND OF THE INVENTION

The introduction of contrast-enhanced ultrasound imaging has significantly expanded the diagnostic potential of ultrasound as a non-invasive and inexpensive imaging modality. Imaging tissue perfusion is feasible using dynamic contrast-enhanced ultrasound (DCE-US) methods in contrast-mode to isolate contrast signal from tissue signals in several abdominal indications, as well as other anatomical sites accessible to ultrasound, and has been adopted in global clinics.

While both CT and MRI imaging modalities also offer contrast-enhanced tissue perfusion measurements, ultrasound-based contrast imaging is advantageous clinically because it is inexpensive relative to other imaging modalities, without radiation, available at the bedside, and offers a uniquely intravascular contrast agent that does not leak out of tumor permeable vessels. More recently, three-dimensional (3D) DCE-US was introduced with the availability of contrast-mode imaging on commercially-available clinical matrix transducers. 3D DCE-US offers volumetric imaging that minimizes sampling errors and operator-influence on quantification. This is particularly important in longitudinal imaging applications such as cancer treatment monitoring. In contrast, imaging tumors via conventional 2D DCE-US is prone to sampling errors and can produce biased quantitative results because of plane-to-plane perfusion variation and tumor heterogeneity. 3D DCE-US imaging mitigates these errors by imaging the tumor as a whole through dynamic volumetric images.

Quantification techniques have been introduced to characterize tissue perfusion properties from DCE-US imaging studies. Perfusion changes, which can be quantified longitudinally using CE-US methods, are indicative of both disease type and treatment response. However, the quality of quantification is affected by several imaging and clinical limitations. In particular, ultrasound-based dynamic imaging is highly susceptible to motion artifacts arising from patient movement, respiration, and peristalsis, as well as operator hand stability. Motion artifacts can be especially hampering in lengthy acquisitions and in the context of quantification by introducing significant errors to the quantified contrast signal.

In the ability of conventional 2D DCE-US imaging to measure tumor response to targeted therapy in renal cell carcinoma patients, significant differences were observed due to respiratory motion in perfusion parameters after bolus time-intensity analysis.

In ultrasound imaging of focal liver lesions technical failures can be encountered due to motion artifacts from patient movement. Motion correction (MC) techniques that account for motion artifacts are essential for reliable usage of DCE-US imaging in cancer treatment monitoring.

While MC algorithms exist for 2D-based DCE-US that take advantage of anatomical accompanying Bmode images over time, conventional 2D DCE-US imaging does not allow for out-of-plane MC, which is a fundamental limitation in attempting to motion correct 2D DCE-US studies. An inability to implement out-of-plane MC is not an issue with 3D DCE-US studies, however, no such side-by-side Bmode images are available in current implementations of 3D DCE-US imaging. This lack of anatomical accompanying Bmode images poses a challenge for using stable anatomical features as a reference for registration and motion compensation. Additionally, the relatively slow frame rates, compared to 2D imaging, and highly dynamic nature of microbubble-contrast agents as they wash-in and -out makes it very challenging to register subsequent frames to one another based off of proximity in time. The need for MC in DCE-US is especially important in quantitative clinical applications where perfusion parameters are used to measure relative changes in blood flow, i.e. treatment monitoring and more so when parametric maps of perfusion parameters are being generated on a voxel-by-voxel basis. The importance of MC in a voxel-by-voxel quantification setting was observed when the inventors analyzed clinical data and noticed that single voxel noise artifacts significantly affects voxel-wise parametric map generation and decreases time-intensity curve quality. The present invention addresses the motion correction need for 3D DCE-US.

### SUMMARY OF THE INVENTION

The present invention provides a validated method for motion correction with great potential for mitigating motion artifacts in 3D DCE-US. In an exemplary embodiment, the inventors demonstrated that the method consistently generates improvement across a variety of different liver metastasis imaging acquisitions qualitatively, and highlights its robustness quantitatively through improvement in lesion volume overlap, increases in image similarity, and provides better accuracy of bolus-time intensity analysis. The results indicate that the utilization of the motion correction method helps to mitigate motion artifacts, thereby improving quantification of 3D DCE-US features and potentially the evaluation of tumor response to treatment and allowing physicians to develop personalized therapies based off treatment response with greater potential for recovery.

In one embodiment, the method is described as a method for motion correction of three-dimensional contrast enhanced ultrasound without the availability of Bmode data. Four-dimensional cine data including three-dimensional contrast enhanced ultrasound image frames are acquired. The acquired three-dimensional contrast enhanced ultrasound image frames are subdivided into groups of similar images referred to as windows. A first pass registration is performed for each of the images in the window to a window representative image. The window representative image is based on the images in that window and could for example be a window average. A second pass is performed for each of the registered images from the first pass to a master reference image. The master reference image is a representation based on all of the acquired three-dimensional contrast enhanced ultrasound image frames and could for example be an average, a maximum intensity, or the like.

In one variation, the invention is embodied as a method for motion correction of three-dimensional contrast enhanced ultrasound without the availability of Bmode data. Four-dimensional cine data including three-dimensional contrast enhanced ultrasound image frames are acquired. The acquired three-dimensional contrast enhanced ultrasound image frames are subdivided into groups of similar images referred to as windows. A first pass registration is performed for each of the images in the window to a window representative image. The window representative image is a function of the images in that window and could for example be a window average, but is not limited to a window average. A second pass registration is performed for each of the registered images from the first pass to a master reference image. The master reference image is a function based on all of the acquired three-dimensional contrast enhanced ultrasound image frames and could for example be an average, a maximum intensity, or the like.

In another variation, the invention is embodied as a method for cine series of three-dimensional contrast enhanced ultrasound images. Four-dimensional cine data including three-dimensional contrast enhanced ultrasound image frames are acquired. The acquired three-dimensional contrast enhanced ultrasound image frames are subdivided into groups of similar images referred to as windows. A first pass registration is performed for each of the images in the window to a window representative image. The window representative image is a function of the images in that window and could for example be a window average, but is not limited to a window average. A second pass registration is performed for each of the registered images from the first pass to a master reference image. The master reference image is a function based on all of the acquired three-dimensional contrast enhanced ultrasound image frames and could for example be an average, a maximum intensity, or the like.

### BRIEF DESCRIPTION OF THE DRAWINGS

FIG. 1 shows an outline of the method according to an exemplary embodiment of the invention. Symbols and their meaning are as follows:

| Symbol | Meaning |
|---|---|
| I | Image Frame |
| n | Frame's time point in imaging |
| | acquisition |
| N | Number of frames in imaging |
| | acquisition |
| A | Imaging acquisition average |
| s | Starting frame for MC |
| g | Frame's window group |
| G | Number of window groups |
| W | Window Average |
| T | Affine transform |
| Ø | Non-Rigid deformation field |

FIG. 2 shows according to an exemplary embodiment of the invention frames from the Pre-MC imaging scan of P-01 (top) compared to their corresponding frames in the Post-MC imaging scan (bottom).

FIG. 3 shows according to an exemplary embodiment of the invention frames from the Pre-MC imaging scan of P-04 (top) compared to their corresponding frames in the Post-MC imaging scan (bottom).

FIG. 4 shows according to an exemplary embodiment of the invention box and whisker plot comparing Pre-MC and Post-MC average lesion volume overlap (left). Side-by-side comparison of two segmented lesions from neighboring frames of two different imaging studies overlaid on one another (right). The two top panels represent segmented lesions from P-01, while the two bottom panels represent segmented lesions from P-04. The left two panels are Pre-MC, while the right two panels are Post-MC. The white segmented lesion is overlapped on top of the red (grey in grey scale) segmented lesion, therefore greater visualization of the red (grey in grey scale) segmented lesion implies greater lesion displacement and poor concordance in tumor morphology between the two frames being compared.

FIG. 5 shows according to an exemplary embodiment of the invention box and whisker plots comparing the average normalized cross-correlation values for the Pre-MC and Post-MC imaging studies. The left plot represents the average normalized cross-correlation values for time frames 35-39, while the right plot represents the average normalized cross-correlation values for time frames 55-59 for each of the 8 patient imaging studies.

FIG. 6 shows according to an exemplary embodiment of the invention a comparison of fitted time-intensity curves at the tumor region of interest for P01 prior to MC (left) and after MC (right).

### DETAILED DESCRIPTION

This invention provides a motion correction technology for 3D DCE-US data unaccompanied by Bmode anatomical imaging that improves the reliability of bolus time-intensity analysis for perfusion parameters. The following is an example of the method and should not be regarded as limiting to the invention.

Materials and Methods

Clinical Case Analysis

The MC method was evaluated using eight human 3D-DCE US imaging studies of liver metastasis. The imaging studies were acquired from patients who provided written consent, were 18 years of age or older, and presented with at least one liver metastasis that was within the range of 1-14 cm in diameter and confirmed using MRI/CT imaging studies. The 3D-DCE US scanning was performed by an experienced sonographer using the commercial Philips X6-1 MHz xMATRIX array transducer at a setting of 1-3 Hz. Each original imaging study (pre-MC) was processed using the method to produce a motion corrected imaging study (post-MC).

Positional Evaluation of MC Improvement

In imaging studies affected by movement, the lesion often changes position within the 3D space captured by the transducer as a result of either patient or operator movement. To quantify the positional shift of the lesion from frame to frame, the lesions (tumors) were manually segmented using ITK-SNAP. Using MeVisLab (MeVis Medical Solutions, Version 3.0) quantification modules, the 3D lesion volume overlap was compared between frames within the sequence to determine the consistency of the lesion's position pre-MC and post-MC.

Similarity Evaluation of MC Improvement

To determine whether MC resulted in a significant improvement that persisted throughout the entire imaging study, image similarity between neighboring frames was quantified within the imaging studies pre-MC and post-MC using normalized cross-correlation.

Bolus Time-Intensity Evaluation of MC Improvement

The presence of motion artifacts impairs the quality and reproducibility of quantitative data when generating time-intensity curves (TIC) for 3D DCE-US imaging studies. When motion artifacts are present, movement of the lesion of interest results in incorporation of noisy and irrelevant voxel intensity data into the TIC quantification software. Consequently, TIC data on tumor perfusion typically provides an inaccurate assessment of tumor response to cancer treatment. To evaluate improvement of TIC reliability, TICs were generated from the imaging studies pre-MC and post-MC using an in-house quantification module written in MeVisLab (MeVis Medical Solutions, Version 3.0) and MATLAB (Math Inc., Version 9.4). The quality and reliability of the TIC was assessed by measuring the sum of squared errors (SSE), the root-mean-squared-errors (RMSE), and R² values and comparing the values measured pre-MC and post-MC.

Motion Correction

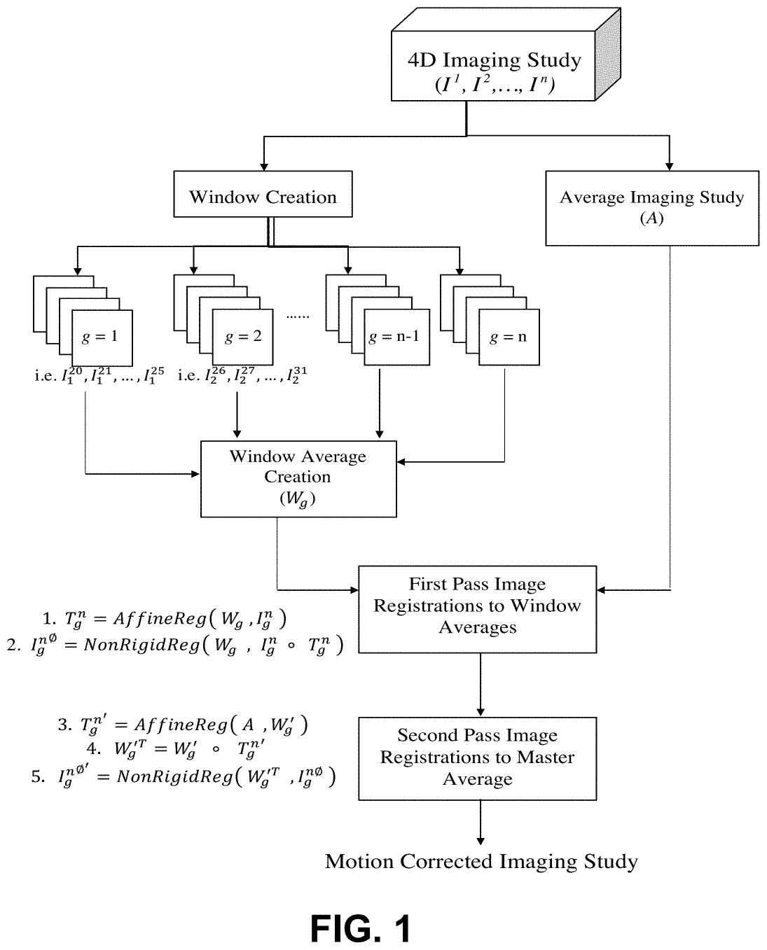

The MC method is based of a two-pass window approach that relies on region-of-interest image masking, employs both affine and non-rigid image registration, and avoids the issue of variability between neighboring images biasing image registrations (FIG. 1). A NiftyReg open-source medical image registration software and the FSL FMRIB Software Library for Imaging Analysis was used in the implementation and executed the method on a Stanford Sherlock Computing Cluster.

Each image acquisition represents a 4D cine having a 3D volumetric US image frames. In each imaging study, there are N image frames, I, including the video, I^{nϵ{1, . . . , N}}. All n images are used to create a master reference image for later use, A, that is the average of all the 3D image frames in the acquisition. The method corrects images following microbubble injection. Since microbubble injection occurs at different frames for each acquisition, the method determines the starting image frame, I^{S}, for MC by searching for the first image with a mean voxel intensity that exceeds 10% of the mean voxel intensity in baseline images. Manual verification was used to ensure that the correct frame was selected for each data set. The images identified for MC, nϵ{1, . . . , N−s}, are then divided into w short temporal windows of equal sizes, W_{gϵ{1, . . . , G}}. Once the images have been categorized into specific windows, I_{w}ⁿ, the images can then proceed to reference creation and image registration. The MC method takes advantage of a series of affine and non-rigid image registrations between reference images and floating images (image that is restructured with respect to the reference image) to spatially realign and eliminate shape distortions within the images.

First Pass

The first pass of image registration is intended to spatially realign and reshape the images with respect to the neighboring images within their respective windows. The initial image should ideally undergo a series of translations, rotations, resizing, shearing, and deformations that make it similar structurally and positionally to its neighboring images. Therefore, a window reference image, W_{g}, is created by averaging all the image frames comprising the window. The first pass of registrations begins with an affine registration of the images, I_{g}ⁿ, to their window average, W_{g}, to create a spatially realigned image, I_{g}ⁿ′. The image frame, I_{g}ⁿ′, then undergoes a non-rigid registration using the affine transformation, T_{g}ⁿ, as an initialization to create the final image product of the first pass, I_{g}^{nø}.

T_{g}ⁿ=AffineReg(W_{g},I_{g}ⁿ) (1)

I_{g}^{nø}=NonRigidReg(W_{g},I_{g}ⁿ·T_{g}ⁿ) (2)

Second Pass

The second pass's purpose is to realign and reshape the image product of the first pass with respect to the entire imaging. Since the first pass created a new series of images, a new window reference image, W_{g}′, is created by averaging all the newly transformed images within the window. The new window averages are then registered using an affine transform to the original master average, A, to create window averages, W_{g}′^{T}, that are all spatially in sync with the average of the entire study. The second pass concludes with a non-rigid registration of the first pass image products, I_{g}^{nø}, to their final window averages, W_{g}′^{T}. The products of the second pass, I_{g}^{nø}′, should now be structurally and positionally similar to both images inside and outside their respective windows, thereby creating a motion corrected image study that occupies the same 3D space throughout the entire sequence.

T_{g}ⁿ′=AffineReg(A,W_{g}′) (3)

W_{g}′^{T}=W_{g}′·T_{g}ⁿ′ (4)

I_{g}^{nø′}=NonRigidReg(W_{g}′^{T},I_{g}^{nø}) (5)

MC Method Technical Details

Image registrations performed in the method were executed by the open-source NiftyReg medical image registration software. Briefly, the rigid registration relied on a symmetric block-matching algorithm and least-trimmed square regression. A 12 degrees of freedom (affine) transform was employed, with a normalized mutual information (NMI) similarity metric and a threshold of 10% outlier blocks in the optimization scheme. For the non-rigid (deformable) b-spline registration, the implementation performed a three-stage approach each with a higher resolution (grid), utilizing NMI similarity with 64 bins, a spline spacing of 5 voxels at the full resolution. For regularization, a bending energy penalty of 0.3 was used to constrain non-realistic deformations, and log of the Jacobian determinant penalty of 0.1. These parameters were optimized using a grid-search on 3 datasets.

To improve the quality of image registration, region-of-interest masks were created using the FSL FMRIB software library and MeVisLab visualization (MeVis Medical Solutions, Version 3.0) to constrain the registration methods to the relevant image features. One mask was constructed per 4D acquisition. The method or software pipeline was designed to be executed by a Stanford Sherlock Cluster, which provides 64000 MB of memory per node and 16 cores per node. The pipeline was rewritten to accommodate parallel processing within the Stanford Sherlock Cluster to decrease lengthy runtimes. Parallelization of the MC algorithm was able to reduce the processing times approximately four-fold on average (720 minutes to 180 minutes).

Results

Positional Improvement Evaluation

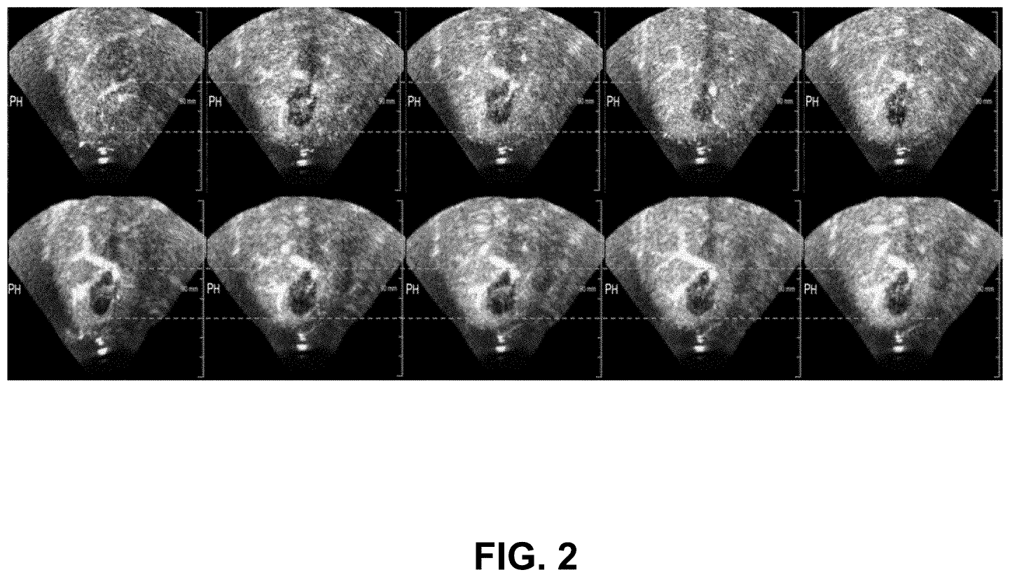

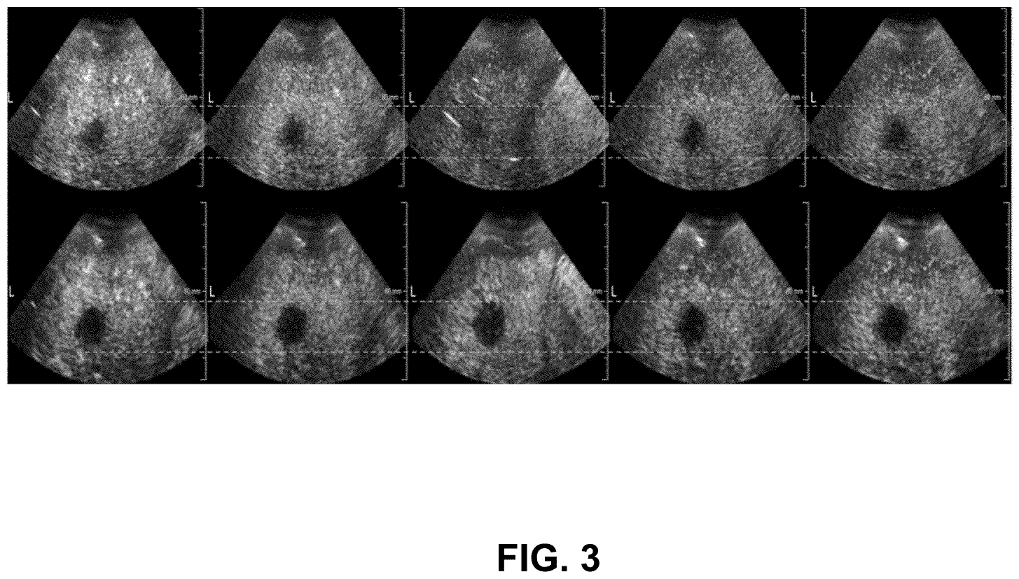

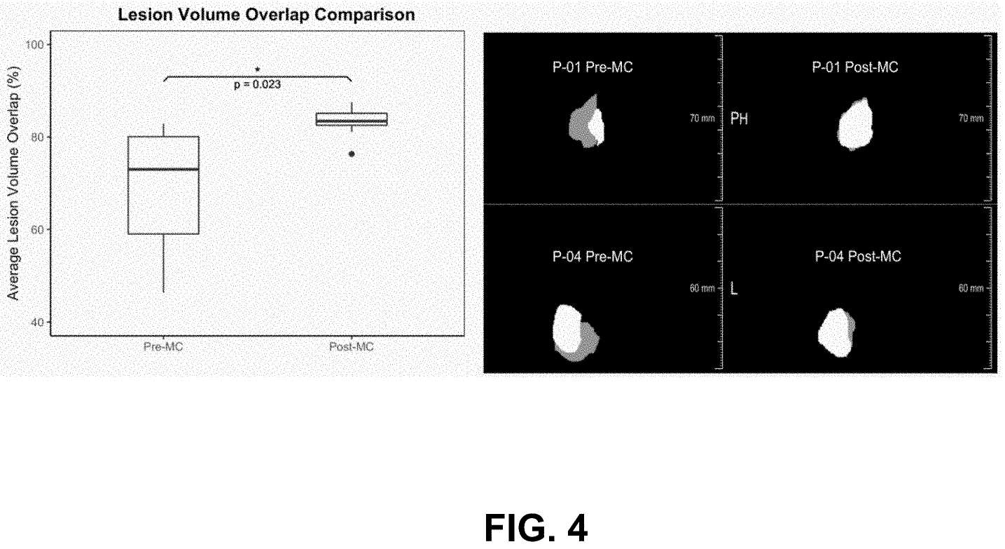

Following MC, the volume overlap of the lesions increased significantly (p=0.023), and the overall lesion remained in the same 3D position (FIGS. 2-3). Table II as referenced in Appendix B in U.S. Provisional Patent Application 62/814,054 filed Mar. 5, 2019, which is incorporated herein by reference, expresses the average percentage of lesion volume that overlaps with the other images in the sample sequence for all eight patient studies. For images that exhibited poor positional similarity and low volume overlap initially (˜40-60%), MC was capable of significantly improving the positional similarity and volume overlap to exceed 80% for most data sets. For images that possessed high-volume overlap prior to MC, the images post-MC either improved slightly or remained at relatively the same volume overlap, indicating that the MC method does not cause regression in images not heavily affected by motion artifacts. FIG. 4 shows the improvement in lesion repositioning following MC.

Image Similarity Improvement Evaluation

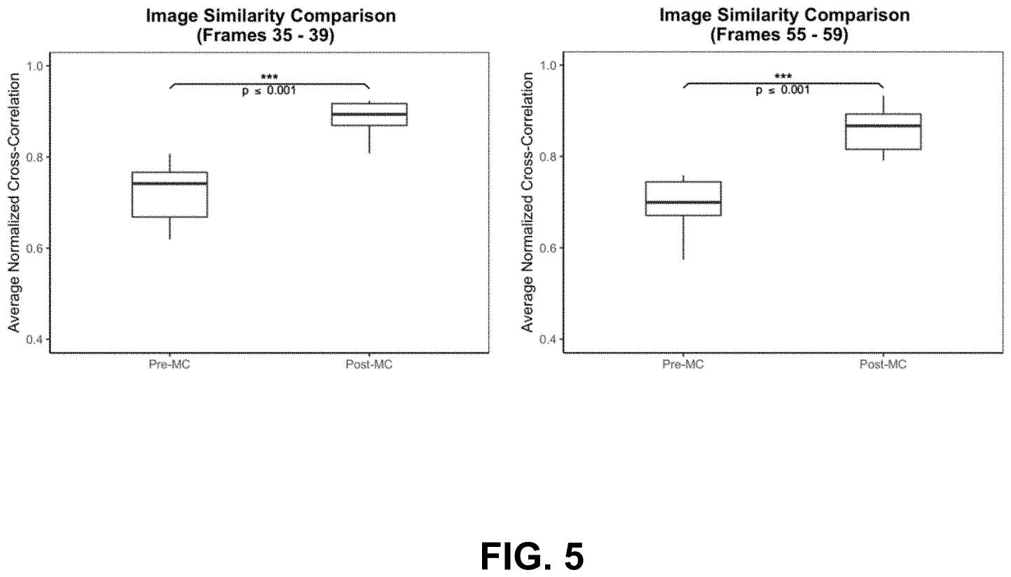

The similarity analysis shows that there is an improvement in normalized correlation coefficients post-MC. Table II as referenced in Appendix B in U.S. Provisional Patent Application 62/814,054 filed Mar. 5, 2019, which is incorporated herein by reference, shows that motion corrected images have higher image similarity, meaning that consecutive images were able to represent similarly positioned anatomy with respect to time following MC. Significant improvement (p≤0.001) in image similarity at two different sets of image frames (35-39 and 55-59) within the same 4D sequence persists throughout the imaging acquisition and not just at specific windows of the 4D sequence as shown in FIG. 5.

Time-Intensity Analysis Improvement Evaluation

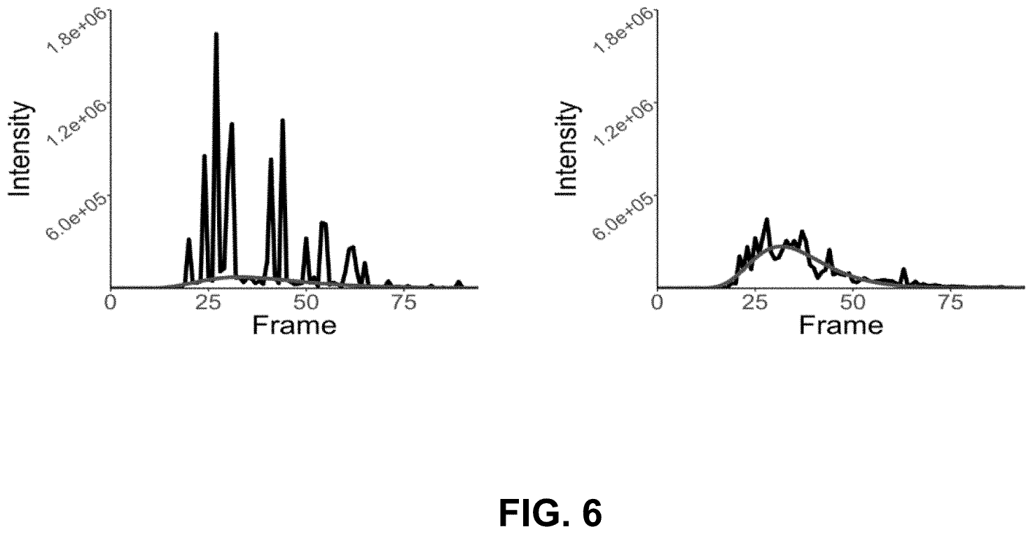

MC reduces the average SSE and RMSE for all the generated TICs, as shown in Table III as referenced in Appendix B in U.S. Provisional Patent Application 62/814,054 filed Mar. 5, 2019, which is incorporated herein by reference. Furthermore, the R² value also increased significantly following MC, indicating that the fitted curve for TICs experienced less noisy and outlier data. The fitted curves for motion corrected data experienced less data spikes qualitatively and a decreased range of intensity values, as shown in FIG. 6.

### CONCLUSIONS

DCE-US imaging is highly susceptible to changes in the imaging window and transducer beam stemming from operator and/or subject movement. In the context of quantitative analysis of volumetric (3D) imaging studies for cancer treatment monitoring, minor changes in the orientation of the transducer can be problematic for the reliability and reproducibility of the perfusion parameters extracted. Studies into quantitative mapping of tumor vascularity have reported errors ranging from 6.4% to 40.3% due to millimeter-sized deviations in transducer positioning. As a result, a MC method was developed herein that aims to mitigate the degree of misalignment found inherently in each pre-processed imaging study.

For the first evaluation methodology (e.g. FIG. 4), volume overlap for a window series of five volumetric imaging frames per patient increased significantly following MC. Additionally, tumor morphology that previously differed qualitatively also improved in similarity with the lesions closely resembling its neighboring frames. This is advantageous during voxel-by-voxel quantification of DCE-US imaging studies for perfusion parameters because it reduces the potential for non-lesion intensity signals to interfere with the region-of-interest and cause the quantification software to misinterpret surrounding noise as relevant perfusion. This is highlighted in one case investigating the quantification of tumor microvascularity where more than 50% of acquisitions were not quantifiable due to excess noise caused by motion that made the lesions non-stationary and the TICs highly sporadic.

Image similarity comparisons using normalized cross correlation were employed to confirm that voxel intensities in one frame corresponded to those of the neighboring frames.

As seen for example in FIG. 5, the magnitude of the average image similarity per patient improved significantly for two different window series, implying that the voxel correlation between subsequent frames in the imaging acquisition has improved. With improved voxel alignment from frame to frame within a volumetric imaging study, an improvement in voxel-by-voxel perfusion quantification should be expected. Due to improved concordance and alignment between voxels going from frame to frame, the maximum intensity values in time-intensity curves should decrease because the interference of misaligned, non-lesion voxel signals in TIC quantification will be mitigated. Furthermore, by decreasing interference from misaligned voxels, the fitted curve to the time-intensity curve should result in a better quality of fit, as measured by R² and the sum of squared errors.

For the time-intensity curve analysis, all of the expected observations were verified following generation of bolus time-intensity curves and quantitative metrics. Following MC for every patient's imaging study, the SSE decreased (p=0.065), while the RMSE error decreased significantly (p=0.018). The R² value for the fitted curve increased significantly (p=0.029). These trends highlight how the MC algorithm improves TIC curve fitting by decreasing the variance in the intensity signals of the imaging acquisition that are used for perfusion quantification. Additionally, for some imaging acquisitions, the maximum intensity observed pre-MC reaches values nearly three-times greater than the maximum intensity observed post-MC, as seen in FIG. 6. This indicates that the MC method is able to improve the reliability of quantification findings by mitigating the number of occurrences where non-lesion intensity values are interpreted as tumor perfusion, vasculature, and angiogenesis.

The advent of DCE-US imaging in the context of clinically monitoring tumor angiogenesis and microvasculature and evaluating response to anti-angiogenic treatment has been fueled by promising results observed in many preclinical and clinical studies investigating the utility of the imaging modality. Preclinical studies have been able to demonstrate that the vascular pathology and tumor microvasculature perfusion determined by CEUS quantification is concordant with the actual histopathology of the tumor, while clinical studies have successfully differentiated between malignant and benign tumors, as well as responding and non-responding tumors to anti-angiogenic therapy, by employing TIC analysis to measure kinetic properties of tumor perfusion and identify morphological differences between tumors. Having verified the qualities of the MC method provided herein in clinical data sets, the method could be applicable in the clinical setting to improve the assessment of tumor response to treatment. Parametric mapping and time-intensity curve analysis have been able to successfully characterize to a limited degree, so it is inventors' hope that the MC method's ability to significantly decrease the poor signal-to-noise ratio within the lesion's ROI, improve the similarity of images on a frame-to-frame basis, and elucidate the tumor's morphology will translate into more accurate and confident prognostications of patient progress during cancer treatment.

Embodiments of the method can be a computer-implemented method executable by computer hardware, a computer code where the methods steps are executable by a computer processor, or a method distributed over an Intranet or Internet whereby the method steps are executed by a computer server.

What is claimed is:

1. A method for motion correction of three-dimensional contrast enhanced ultrasound without the availability of Bmode data, comprising:
(a) acquiring four-dimensional cine data including three-dimensional contrast enhanced ultrasound image frames;
(b) subdividing the acquired three-dimensional contrast enhanced ultrasound image frames into groups of similar images referred to as windows;
(c) registering as a first pass each of the images in the window to a window representative image, wherein the window representative image is based on the images in that window; and
(d) registering as a second pass each of the registered images from (c) to a master reference image, wherein the master reference image is a representation based on all of the acquired three-dimensional contrast enhanced ultrasound image frames.

2. The method as set forth in claim 1, wherein the window representative image is a window average, wherein the window average is based on the images in that window.

3. The method as set forth in claim 1, wherein the representation is an average or a maximum intensity based on all of the acquired three-dimensional contrast enhanced ultrasound image frames.

4. A method for motion correction of three-dimensional contrast enhanced ultrasound without the availability of Bmode data, comprising:
(a) acquiring four-dimensional cine data including three-dimensional contrast enhanced ultrasound image frames;
(b) subdividing the acquired three-dimensional contrast enhanced ultrasound image frames into groups of similar images referred to as windows;
(c) registering as a first pass each of the images in the window to a window representative image, wherein the window representative image is a function of the images in that window; and
(d) registering as a second pass each of the registered images from (c) to a master reference image, wherein the master reference image is a function based on all of the acquired three-dimensional contrast enhanced ultrasound image frames.

5. The method as set forth in claim 4, wherein the window representative image is a window average, wherein the window average is based on the images in that window.

6. The method as set forth in claim 4, wherein the representation is an average or a maximum intensity based on all of the acquired three-dimensional contrast enhanced ultrasound image frames.

7. A method for registering a cine series of three-dimensional contrast enhanced ultrasound images, comprising:
(a) acquiring four-dimensional cine data including three-dimensional contrast enhanced ultrasound image frames;
(b) subdividing the acquired three-dimensional contrast enhanced ultrasound image frames into groups of similar images referred to as windows;
(c) registering as a first pass each of the images in the window to a window representative image, wherein the window representative image is a function of the images in that window; and
(d) registering as a second pass each of the registered images from (c) to a master reference image, wherein the master reference image is a function based on all of the acquired three-dimensional contrast enhanced ultrasound image frames.

8. The method as set forth in claim 7, wherein the window representative image is a window average, wherein the window average is based on the images in that window.

9. The method as set forth in claim 7, wherein the representation is an average or a maximum intensity based on all of the acquired three-dimensional contrast enhanced ultrasound image frames.
